(19) United States
(12) Patent Application Publication
(10) Pub. No.: US 2011/0190776 A1
(43) Pub. Date: Aug. 4, 2011

(54) INTEROSTEAL AND INTRAMEDULLARY IMPLANTS AND METHOD OF IMPLANTING SAME

(75) Inventor: Julio C. Palmaz, Napa, CA (US)

(73) Assignee: Palmaz Scientific, Inc., Dallas, TX (US)

(21) Appl. No.: 12/973,854

(22) Filed: Dec. 20, 2010

Related U.S. Application Data
(60) Provisional application No. 61/288,225, filed on Dec. 18, 2009.

(51) Int. Cl. A61B 17/58

(52) U.S. Cl. 606/92

(57) ABSTRACT

The present invention relates to an expandable metal balloon that may be used for the treatment of diseased or injured bone tissues, and a method of using the same. The metal balloon is inserted into the interior space of a cancellous bone tissue, and is filled with a suitable material to provide internal structural support to the bone.

### CROSS-REFERENCE TO RELATED APPLICATIONS

The present application claims priority from U.S. Provisional Patent Application Ser. No. 61/288,225, filed on Dec. 18, 2009, which is hereby incorporated in its entirety.

### FIELD OF THE INVENTION

The present invention relates generally to intra- and interosteal implants of the type intended to support bony structures. More particularly, the present invention relates to intra- and interosteal implantable devices that conform to and support an intra- or interosteal geometry.

### BACKGROUND OF THE INVENTION

There is presently known in the art a wide range of treatments for diseased or injured cancellous bone tissues in mammals. Cancellous, or spongy bone, has a trabecular (honeycomb structure) and a high level of porosity relative to cortical bone. The spaces between the trabeculae are filled with red bone marrow containing the blood vessels that nourish spongy bone. Spongy bone is found in bones of the pelvis, ribs, breastbone, vertebrae, skull, and at the ends of the arm and leg bones.

Bone related disorders affect numerous individuals and, if treatable, often require long periods of treatment and highly invasive measures, which often causes significant pain and discomfort. Many individuals, due to various reasons, are prone to bone fractures or breaks and often require preventive measures to reduce this risk. Also, many individuals experience fractures or breaks that fail to heal within a normal time period without some kind of intervention or treatment. These various complications require medical intervention to help prevent or repair bone disorders.

Known methods for fixing or repairing broken bone are invasive measures involving the insertion of a long, narrow nail or rod through the separated bone segments. This physically connects the separated bone segments and braces the segments together to promote healing. In order to accomplish this method, often, the bone medulla must be reamed out before insertion of the rod. One specific method calls for an intramedullar nail with an expanding mechanism. The nail includes an outer tubular sheath, a rod-shaped element longitudinally movable in the sheath, and an expandable element having two or more spreadable longitudinal branches at the front end of the nail. The nail is inserted into the medullar cavity of a bone with the end of the nail protruding out of the end of the bone. The rod-shaped element, when pulled back, causes the branches of the expander element to spread radially outward, thereby anchoring the front end of the nail within the intramedullar cavity. One specific example of a fracture that requires medical intervention to help heal is a nonunion fracture, which is a fracture that fails to heal without intervention. Current forms of treatment for nonunion fractures include electrical stimulation, bone grafting, and internal fixation (as mentioned above using rods).

Known methods of repairing degenerative bone, or adding support to bone, e.g., vertebrate, include inflatable or conformable implantable devices. Conformable implantable devices are conventionally used in bone fracture fixation and vertebroplasty. Conventional methods of vertebroplasty often employ a polymerizable material that is delivered into the intervertebral space and polymerized in situ to fill an intervertebral space. It is also know to inject a polymerizable material into a void pace within a bone in order to fill the void space and support bone regrowth. However, the conventional methods fail to contain the polymerizable material into a constrained geometry that conforms to the void space being filled. By failing to contain the injected material, spillage of the injected material into veins, spinal canal, neural foamina and other critical anatomical structures often occurs, allowing seepage of unwanted material into anatomical locations where it is undesirable. Solidifiable polymers have been found to exhibit some chronic inflammatory or carcinogenic potential as long term implant materials. Therefore, it is desirable to fashion a means for confining a solidifiable material in a manner that permits the solidifiable material to conform to a void space being filled in situ.

In one specific example, disclosed in U.S. Pat. No. 6,235,043, an inflatable balloon is used to support or fix bone structure. The disclosed balloon is made of a non-expandable but flexible material, specifically PET or Kevlar. Also, the balloon is constructed so that upon nearly full inflation, it forms a predetermined shape dictated by the bone cavity it was made to fill. The inflated balloon acts by compressing cancellous bone to create a cavity in the bone and restore the original position of the outer cortical bone that was fractured or collapsed. The problems arising from this balloon-like device is that the bone support is derived from the material used to inflate the balloon, which exerts high pressure on the balloon itself. This increases the risk of leakage of filling material which is often inflammatory or carcinogenic. Also, efficient support of bone structure is limited and often complicated because the shape of the balloon must be predetermined before application of the balloon into the bone cavity. It would be preferable to insert an expandable device that can assume the shape of the cavity in situ.

There still remains a need for treating or supporting degenerative, fractured or broken bone in a mildly invasive manner. Accordingly, there still remains a need for a device that can fix or support bone structure in a mildly invasive manner.

All bones are subject to damage by trauma, disease processes, or fractures, such as, but not limited to, osteoporosis, osteoporotic bone, osteoporotic fractured metaphyseal and epiphyseal bone, osteoporotic vertebral bodies, fractured osteoporotic vertebral bodies, fractures of vertebral bodies due to tumors, especially round cell tumors, avascular necrosis of the epiphyses of long bones, especially avascular necrosis of the proximal femur, distal femur and proximal humerus, defects arising from endocrine conditions, and metastatic tumors. The bones comprising the vertebral spine are particularly difficult to treat due to the complexity of their anatomical structure. Effective treatment of the vertebra is further exacerbated by the proximity of the spinal cord to the nerves emanating therefrom.

Two minimally invasive procedures that have gained popularity in the treatment of fractured or diseased bones, and in particular the vertebra, are percutaneous vertebroplasty and Kyphoplasty. U.S. Pat. No. 6,273,916 describes a method and apparatus for performing vertebroplasty. Vertebroplasty is a procedure wherein a cement-like material, such as polymethylmethacrylate (“PMMA”), is injected under high pressure directly into the vertebral cavity. The cement-like material is permitted to cure, and upon hardening, provides structural support to the affected vertebra.

In Kyphoplasty, a small incision is made in the back. Using fluoroscopic imaging techniques, a surgeon guides a cannula to a desired position, inserts a drill through the cannula, and bores through the cortical wall into the cancellous bone to define a channel within the vertebral body. The drill is removed and a balloon catheter is inserted into the channel. The balloon catheter is then inflated to compress the cancellous bone against the inner cortical wall to define a cavity therein. A particular advantage of this procedure for compression fractures is that inflation of the balloon catheter restores a portion of the vertebral height. Following deflation and removal of the balloon catheter, a cement-like material, such as that used in vertebroplasty, is injected to fill the cavity. The cement is permitted to cure, and the surgical site is closed.

Variations of percutaneous vertebroplasty and Kyphoplasty are known in the prior art. For example, U.S. Pat. No. 5,827,289 discloses using a balloon to form or enlarge a cavity or passage in a bone, especially in, but not limited to, vertebral bodies and to deliver therapeutic substances to bone in an improved way. U.S. Pat. No. 6,632,235 discloses using inflatable devices for reducing fractures in bone and treating the spine. U.S. Patent Application Publication No. US 2003-0050644 A1 discloses employing an expandable body that is inserted into bone over a guide wire. U.S. Patent Application Publication No. US 2005-0234456 A1 discloses using an implantable medical device for supporting a structure. U.S. Pat. No. 6,348,055 discloses using a conduit for delivering an implant material from a high pressure applicator to an implant delivery device. U.S. Pat. No. 6,033,411 discloses using precision depth-guided instruments to perform percutaneous implantation of hard tissue implant materials.

While the aforementioned procedures represent significant advances in the treatment of bone injuries and diseases, they are not without risk. A risk common to both procedures is the exfiltration of the cement from a fracture site in the treated bone. While these risks are more pronounced in vertebroplasty, due to the high injection pressures, exfiltration of the cement from the fracture site can lead to thrombosis, spinal stenosis, or nerve root compression, and in rare cases pulmonary embolus.

A further limitation of the aforementioned procedures is that once the bone cement has cured, subsequent removal of the cement from the vertebral body is prohibitive, particularly in the case of vertebra in the spine.

Similarly, the aforementioned methods are reparative and make no provision for the treatment of any underlying disease condition which may have caused or contributed to the fractures necessitating the application of these methods in the first place.

Accordingly, despite these recent advances in the art, there remains a continuing need for improved devices and methods for treating bone fractures and disease conditions.

### SUMMARY OF THE INVENTION

The present invention is directed to a device that can be used to treat or prevent a variety of bone-related complications or bone disease. The device acts as a physical means of supporting or fixing bone in a long-term manner, thereby acting to provide extra support to existing bone structure to help relieve the stress caused by degeneration of bone, including breaks or fractures. Furthermore, the supporting feature of the device can be extended to musculoskeletal and soft tissue structures other than bone, such as cartilaginous tissue.

The present invention is also directed to a method of treating diseased or injured bone tissue comprising selecting an interior area in a bone tissue to be treated, inserting a device into the interior area of the bone tissue to be treated, and internally supporting the bone tissue using the device during treatment.

Generally, the device consists of a metallic balloon capable of expanding within bone matter. The metallic balloon defines an enclosure with at least a single point of ingress and/or egress of a filler material into the enclosure. The geometry of the metallic balloon is preferably selected to correspond to the intended medical application of the invention. For example, an intervertebral balloon will have a geometry corresponding to the geometry of an intervertebral space anatomically occupied by an intervertebral disc. As for an intramedullary implant, such as for example, fixation of long bone fractures, the implant will generally have an elongated cylindrical shape that can reside within the marrow canal of the long bone. Alternatively, depending upon the fixation required, a canal may be formed, such as by drilling into the bone, and the intramedullary implant positioned within the formed canal. With regard to short bones, the balloon is typically implanted so it expands within trabecular tissue. If required, a section of bony tissue can be opened for the insertion of the metallic balloon. Those of ordinary skill in the art will understand that the geometry of the inventive metallic balloon is limited only by the anatomical constraints imposed by the intended use of the implant.

The device consists of a metallic balloon that is implantable within bony structure without significant risk of generating an immune response or causing rejection by the host. As the name suggests, the balloon is made of a metallic material that is biocompatible and has the capacity to expand outward upon introduction of an outward force. Upon expansion, the balloon generally conforms to the shape of the cavity in which it is implanted and can withstand or support pressure generated by the bony structure directed into the cavity. The metallic balloon is made to expand upon the addition of filler material, e.g., gas, including nitrogen and inert gas, or a solidifiable liquid. The addition of filler material provides the outward force needed to expand the metallic balloon and provides force outward against bony matter, which acts to support the bony structure. Because the metallic balloon itself is able to provide some support against bony structure, the metallic material reduces the amount of pressure required to be generated by the filler material, which, if high enough, increases risk of leakage of the filler material. The filler material can be a gas, including nitrogen and inert gases, or a solidifiable liquid, including acrylic, urethane and silicon polymers. Typically, the metallic balloon is made from nitinol using various fabrication techniques, preferably vacuum deposition. Additionally, the metallic balloons have a titanium oxide surface that helps bone-tissue compatibility and reduces risk of rejection by the host.

One aspect of the invention is the method employed for use of the metallic balloon. Metallic thin films having highly controlled material and mechanical properties may be formed in highly varied geometric shapes by vacuum deposition techniques, such as physical vapor deposition or chemical vapor deposition. Physical vapor deposition techniques suitable for use in fabricating the metallic balloon of the present invention are more fully described with reference to U.S. Published Patent Application No. 20040181252, published Sep. 16, 2004 10/693,572, U.S. Pat. No. 6,733,513, U.S. Published Patent Application No. 20030059640 published Mar. 27, 2003 or U.S. Pat. No. 6,379,383, each of which is hereby incorporated by reference. Chemical vapor deposition techniques suitable for use in fabricating the metal balloon of the present invention are more fully described with reference to U.S. Published Patent Application No. 20070061006, published Mar. 15, 2007, which is also hereby incorporated by reference.

In one aspect of the current invention, a device for treating diseased or injured bone includes a catheter, wherein the catheter comprises a main body defining at least one interior passage therethrough. The device further includes an expandable deformable structure, wherein the expandable deformable structure defines an interior space. The device also includes a fastener that removably connects the catheter to the expandable deformable structure, wherein the improvement comprises the expandable deformable structure comprised of a metallic material.

In another aspect of the current invention, a minimally invasive medical device includes a deformable structure expandable from a first reduced geometric state to a second expanded geometric state and having a fillable chamber within the deformable structure. The device further includes a conduit communicating with the fillable chamber, and a hardenable filler material capable of being introduced through the conduit and into the fillable chamber. The second expanded geometric shape is supported by the hardenable filler material, wherein the improvement comprises the deformable structure comprising a metal balloon.

In yet another aspect of the current invention, a method of treating diseased or injured bone tissue includes the steps of selecting an interior area in a bone tissue to be treated and inserting a geometrically expansive metal balloon into the interior area of the bone tissue to be treated. The method further includes the steps of expanding the metal balloon to a desired expanded three dimensional geometry and fixing the desired expanded geometry thereby internally supporting the bone tissue using the device during treatment.

### BRIEF DESCRIPTION OF THE FIGURES

FIG. 1 is a side elevational view of a detachable deformable structure and catheter according to one embodiment of the invention inserted into a cavity defined in the cancellous bone of a vertebra.

FIG. 2 is a side elevational view of the detachable structure and catheter of FIG. 1 as expanded by a bone supporting material.

FIG. 3 is a side elevational view of decoupling of the catheter of FIG. 1 from the deformable structure of FIG. 1 and sealing of the inventive structure structure.

FIG. 4 is a side elevational view of the inventive structure after implantation in a vertebra.

FIG. 5 is a transverse cross-sectional view of a spinal vertebra and arthroscopic probe inserted therein.

FIG. 6 is a transverse cross-sectional view of a spinal vertebra having an inventive structure and arthroscopic probe that is partially inserted into a vertebral body.

FIG. 7 is a transverse cross-sectional view of a spinal vertebra having an inventive structure and arthroscopic probe that is fully inserted into a vertebral body.

FIG. 8 is a side elevational view of an embodiment of a catheter and detachable structure adapted for intramedullary fixation in accordance with the present invention.

FIG. 9 is a side elevational view of a catheter and detachable structure in its expanded intramedullary fixation state in accordance with the present invention.

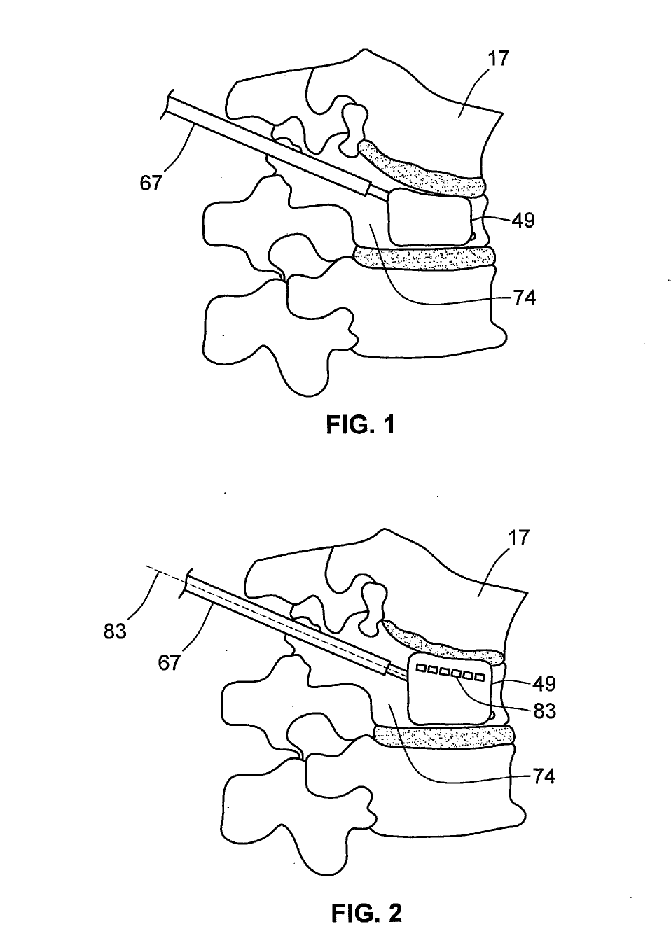

### DETAILED DESCRIPTION OF THE PREFERRED EMBODIMENTS

The present invention relates to the field of orthopedic surgical devices and techniques. The method of treatment of the present invention involves using a catheter 67 that is connected to a preferably detachable deformable structure by a removable fastener 69. The removable fastener may be a screw device, a bayonet type or other interlocking fitting. The fastener 69 releasably connects the catheter 67 to the deformable structure 49 and is capable of coupling the deformable device 49 to the catheter 67 and decoupling the deformable device 49 from the catheter 67.

The catheter 67 has a main body defining at least one interior passage therethrough, the deformable structure 49 defines an interior space, and the deformable structure 49 comprises a sealable port that allows for communication between the interior passage of the catheter and the interior space 51 of the deformable structure 49. Interior space 51 forms a fillable cavity into which a hardenable filler material 83 may be introduced through the catheter 67 to retain an inflated shape of the deformable structure 49.

“Deformable structure” is defined herein as a malleable, expandable, non-rigid structure. The term is more specifically defined as a structure that has a specific compliance rate of about 0% to about 30%. It should be understood that such rate is non-limiting to the scope of the invention. The compliance rate of the deformable structure is defined as the rate at which the structure yields to pressure or force without disruption, or an expression of the measure of the ability to do so, such as an expression of the distensibility of the deformable structure, in terms of unit of volume change per unit of pressure change, when it is filled with liquids or other materials.

The deformable structure may be temporarily or permanently inserted in an interior area such as a cavity or other space within diseased or injured cancellous bone tissue of a mammal in order to internally support the bone and/or to treat such diseases or injuries, and to alleviate symptoms of such diseases or injuries, such as back pain. The detachable deformable structure expands upon introduction, typically by injection, of a suitable bone supporting material, through a passage within the catheter, and the deformable structure provides containment and maintenance of the bone supporting material therein. The detachable deformable structure is preferably shaped such that upon expansion, the structure will generally adapt and conform three-dimensionally to the dimensions of the exterior area such as a cavity defined within the internal cortical walls of the bone to be treated. The detachable deformable structure prevents the exfiltration of the bone supporting material from the fracture site through use of a preferred semi-permeable membrane, and facilitates controlled drainage from the structure, thereby avoiding the deleterious effects described herein above.

To provide additional containment and maintenance of the bone supporting material within the structure, the structure may be provided with a sealable port, through which the catheter communicates with the deformable structure. The port may be sealed upon detachment of the catheter to prevent the bone supporting material from exuding from within the structure. This arrangement further facilitates pressurized containment and maintenance of the bone supporting material within the structure. The port may remain open, but where the bone supporting material hardens and so cannot exude from the port. In another embodiment, the port may be temporarily sealed so that the catheter can be reattached to the port, and the bone supporting material can be removed as necessary.

The deformable structure may be formed from any suitable biocompatible material that is malleable and durable, such as, but not limited to, stainless steel, titanium, polymers such as, for example, polymeric materials and plastics such as polyester and polyethylene, polylactic acid and copolymers of these polymers with each other and with other monomers, resorbable synthetic materials such as, for example, suture material, Nitinol, or any other suitable material as known to those of skill in the art, including combinations of such materials. The suitable biocompatible material is preferably in the form of a thin metallic film material that is super-elastic and possesses excellent rubber-like shape retention. Nitinol, a metal alloy of nickel and titanium, is a particularly suitable biocompatible material because Nitinol has the ability to withstand the corrosive effects of biologic environments, such as that inside cancellous bone tissue. In addition, Nitinol also has excellent wear resistance and shows minimal elevations of nickel in the tissues in contact with nitinol. Betz et al., Spine, 28(20S) Supplement: S255-S265 (Oct. 15, 2003). The use of a suitable Nitinol as a preferred biocompatible material in implantable balloons is disclosed in U.S. Pat. No. 6,733,513, which is incorporated herein by reference.

The deformable structure is preferably in the form of an expandable three-dimensional balloon. Where the deformable structure is permanently inserted into cancellous bone tissue, the biocompatible material of the structure is made of a suitable surface material, such as, but not limited to those mentioned above, to provide a bone-friendly membrane for incorporation and healing and to help improve or accelerate the attraction of healthy bone cells.

In applications where disease is the underlying cause of the bone fracture, an object of the present invention further contemplates that the deformable structure serve as a carrier for a treatment for a disease or injury. The invention contemplated herein includes medicinal, radiological and thermal treatments for the underlying disease conditions. Such medical treatments may include, but are not limited to, such treatments comprising drugs such as, but not limited to, Cisplatin, Taxol.™., Adriamycin.™., Doxorubicin, Melphalan, Cyclophosphamide, Carboplatin, Methotrexate, or similar treatments known to those in the art for treating bone diseases. Such radiological treatments include, but are not limited to, radiation therapy which can be used for treatment of malignant bone disease to prevent further fractures and pain, or interventional procedures which can be applied to malignant bone disease by means of embolization (transvascular occlusion).

The bone supporting material may include a number of materials that are selected based on the purpose of the treatment. Where the treatment encompasses permanent bone support, the bone supporting material includes bone cement that may be injected as a liquid and then which hardens within a short period of time. Where the treatment encompasses temporary support of the bone, the bone supporting material may be injected as a liquid, and will remain a liquid form during the time required for support. It can then be readily withdrawn when the treatment procedure is complete and/or replaced if additional treatment is needed. In alternative embodiments, the bone supporting material may be in the form of a pliable gel-like material to provide support and energy attenuation for the bone structure.

As may be seen in reference to the various drawings, the present invention includes a catheter 67 having at least one lumen or other long extending passage way, preferably a multi-lumen catheter 67, with a detachable deformable structure 49 for temporary or permanent placement in a cavity 74 defined in bone tissue such as cancellous bone tissue 17. The present invention further comprises methods of treating bones which have been fractured through trauma or through disease processes, such as, but not limited to, osteoporosis, osteoporotic fractured metaphyseal and epiphyseal bone, osteoporotic vertebral bodies, fractures of vertebral bodies due to tumors, especially round cell tumors, avascular necrosis of the epiphyses of long bones, especially avascular necrosis of the proximal femur, distal femur and proximal humerus and defects arising from endocrine conditions, metastatic tumors, long bone (i.e., traumatic or spontaneous bone fractures or other local distortions of bone structures), such as cervical, thoracic, lumbar, and sacral fractures, and the like.

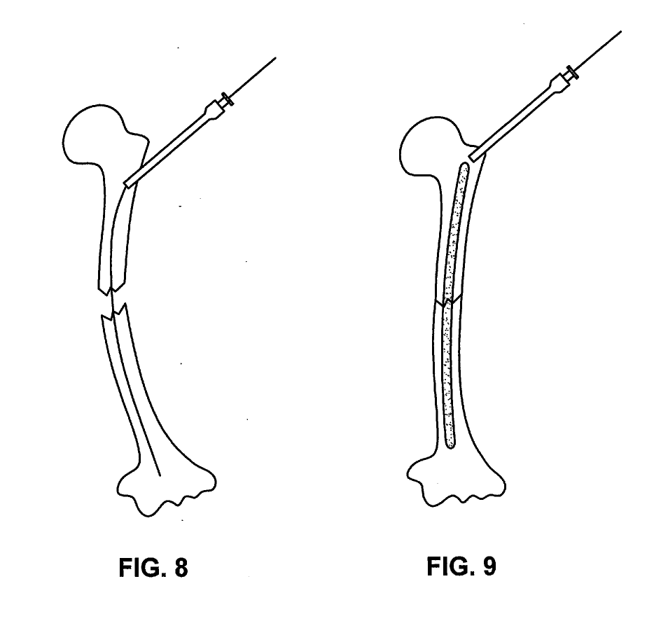

The detachable deformable structure 49, as best shown in FIGS. 6 and 7, is shaped such that it generally conforms to dimensions of a cavity 74 selected within the internal cortical walls of the cancellous bone tissue 17. The cavity 74 may be simply identified and/or defined within the internal cortical walls by any suitable procedure familiar to those of skill in the art, such as, but not limited to, drilling, insertion of a precursor inflatable device, and other related methods. The dimensions of the cavity 74 may be predetermined using minimally invasive image-guided techniques such as, but not limited to, X-ray, CT scan or intraoperative CT imaging, ultrasound, computed tomography, MR/CT image registration, three dimensional visualization, optical localization, and magnetic resonance imaging (MRI), or any other suitable imaging techniques. Preferably, the walls of the deformable structure 49 have a compliance rate of about 10% to about 30%, to provide engagement of the structure with the cavity 74 walls comprising either cancellous bone 17 or the internal walls of the cortical bone.

As depicted in FIG. 2, the detachable deformable structure 49 is expandable upon injection of a suitable bone supporting material 83 through a lumen of the multi-lumen catheter 67, with the structure 49 providing containment and maintenance of the bone supporting material 83 therein and additional structural support to the cancellous bone tissue 17. The characteristics of the bone supporting material 83 are selected based upon whether the structure 49 will be a permanent implantation or whether the structure 49 will be temporarily implanted for a sufficient duration to permit a bone fracture to heal.

For permanent implant treatments, the bone supporting material 83 may be a cement-like material made of a formulation known or to be developed in the art, such as those based on polymethylmethacrylate (“PMMA”), or other suitable biomaterial alternatives or combinations, including, but not limited to, dextrans, polyethylene, carbon fibers, polyvinyl alcohol (PVA), or poly(ethylene terephthalate) (PET), such as those used in conventional vertebroplasty or Kypohplasty procedures. More preferably, the cement-like material is PMMA. Specific formulations of PMMA are known in the art and are commonly used in bone implants. Such formulations include, but are not limited to those disclosed in, for example, U.S. Pat. Nos. 4,526,909 and 6,544,324, which are incorporated herein by reference.

One of the primary objects of the present invention is to prevent exfiltration of the cement-like material from the fracture site and its resulting physiological risks. This prevention is possible due to the containment and maintenance of the cement-like material within the deformable structure 49.

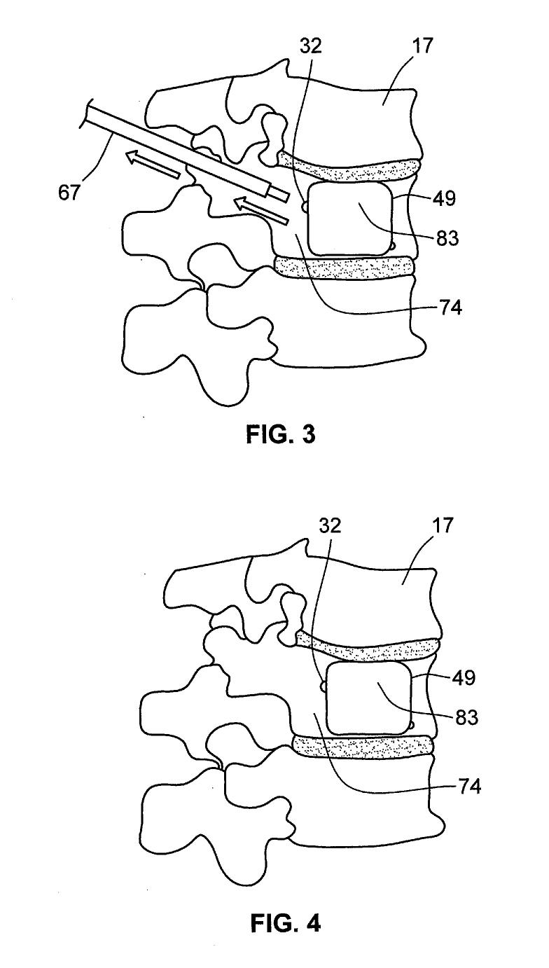

To provide additional containment and maintenance of the bone supporting material 83 within the deformable structure 49, the structure 49 may be provided with a sealable port 32, as shown in FIGS. 3 and 4, through which the catheter 67 communicates with the deformable structure 49. The port 32 may be sealed upon detachment of the catheter 67 to prevent the bone supporting material 83 from leaching out of the structure 49. This arrangement further facilitates pressurized containment and maintenance of the bone supporting material 83 within the structure 49. Additionally, a sealable port 32 also prevents the infiltration of biologic fluids into the deformable structure 49, thereby improving the structure's durability by preventing corrosion and degradation of the walls of the internal deformable structure 49. Alternatively, the catheter 67 may be left attached to the deformable structure 49 until such time as the bone supporting material 83 has cured. Such curing time generally takes about 2 to about 10 minutes if PMMA is used as the bone cement. Once the PMMA has cured, the catheter 67 may then be detached with minimal risk of the material leaching from the sealable port 32, as shown in FIG. 3. The reverse arrows in FIG. 3 from the bone tissue 17 indicate the direction in which the catheter 67 moves after injection of the bone supporting material 83 into the deformable structure 49 and decoupling therefrom. However, because this process potentially leaves the structure 49 temporarily open, care should be taken to the extent necessary, to avoid infiltration of the biological fluids into the structure 49. FIG. 4 depicts the position of the deformable structure 29 after detachment of the catheter 67, leaving only the sealable port 32 attached to the deformable structure 29.

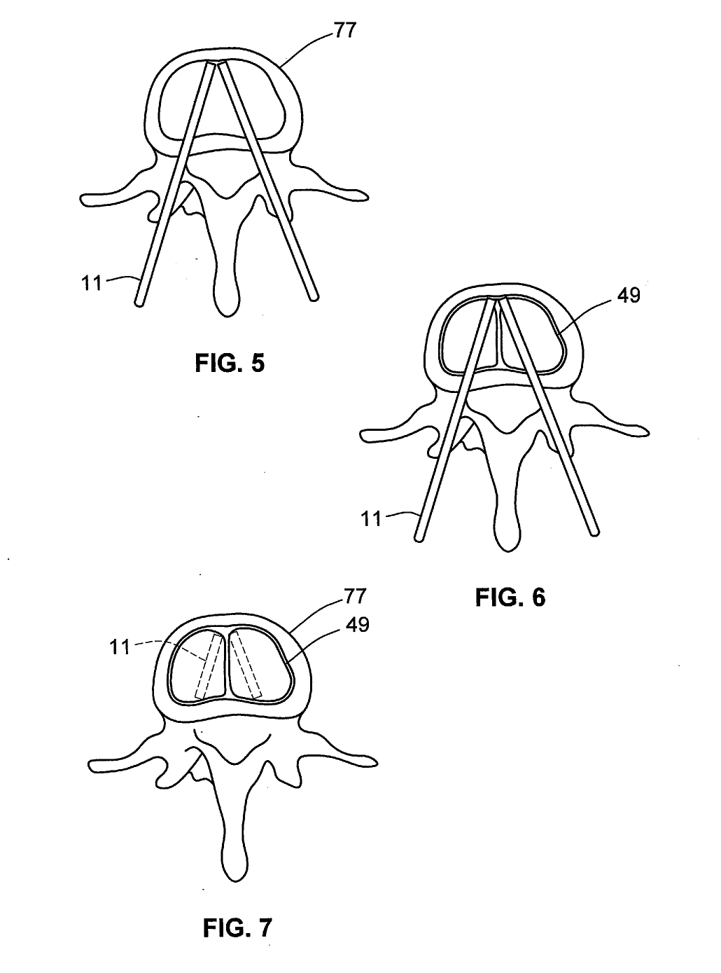

FIGS. 5-7 depict top views of the side-by-side placement of two deformable structures 49 within an intravertebral space of a vertebral body 77 by introducing the deformable structures 49 through catheters 11, inflation of the deformable structures 49 by introduction of a hardenable material into interior space within the deformable structures 49 and the subsequent detachment of the catheters 11.

The device of the present invention may also be utilized for temporary implantation in cancellous bone 17, potentially offering a more advantageous bone setting technique compared to contemporary procedures which rely on insertion of metallic rods, pins or screws to maintain a bone's structure while the fracture is permitted to heal. In this instance the deformable structure 49 would likely require a port having a valve to maintain the strength and rigidity of the structure while the fracture heals, but to allow access to the bone supporting material 83 for evacuation at a later time. In this instance the sealable port 32 also provides for reattachment of the catheter 67 to permit removal of the bone supporting material 83 and extrication of the structure from the bone 17.

The characteristics of the bone supporting material 83 are selected such that it assumes a rigid or semi-rigid state while the bone is healing and is capable of being dissolved, melted, or otherwise withdrawn from the deformable structure 49 once the healing processes have progressed to a point where internal support is no longer necessary. Once the bone supporting material 83 is evacuated from the deformable structure 49, the structure 49 may then be extricated from the bone to permit final healing of the bone 17. An advantage of the deformable structure 49 over that of metallic rods or pins is that its compliance will facilitate its removal with minimal trauma to the cancellous bone 17 as it is extricated.

The deformable structure 49 may be formed from any suitable biocompatible material, such as, but not limited to, stainless steel, titanium, polymers such as, for example, polymeric materials and plastics such as polyester and polyethylene, polylactic acid and copolymers of these polymers with each other and with other monomers, resorbable synthetic materials such as, for example, suture material, Nitinol, or any other suitable material as known to those of skill in the art, including combinations of such materials. Preferably, the deformable structure 49 will be formed from a biocompatible metallic film material, appropriately shaped to generally conform or adapt to a cavity 74 defined in the internal structure of the bone 17 selected for treatment.

An alloy of Nickel and Titanium, commonly known as Nitinol, is well suited to this application, as a result of its proven biocompatibility and its ability to withstand the corrosive effects of biologic environments. Other desirable properties for the metallic film material, and Nitinol in particular, are its super-elasticity and shape memory, which facilitates insertion of the catheter 67 into the cavity 74 defined in the cancellous bone 17. For example, the catheter 67 and the deformable structure 49 made from Nitinol may be inserted into the cavity 74 with the deformable structure 49 in a Martensitic unexpanded state. Once positioned as desired, a relatively warm fluid may be introduced into the deformable structure 49 to raise the temperature of the Nitinol to the phase transition temperature of the Nitinol, thereby causing the Nitinol to expand to an Austenitic state to fill the cavity 74 as desired. The Nitinol deformable structure 49 may subsequently be collapsed for removal by introducing a relatively cold fluid thereinto to lower the temperature of the Nitinol to below phase transition temperature. Moreover, Nitinol's stress-strain characteristics make it an excellent choice to provide additional structural support to the bone 17 in combination with the bone supporting material 83.

For bone treatments encompassing permanent placement of the structure 49, the biocompatible material is provided with a suitable surface treatment to provide a bone-friendly matrix for incorporation and healing within the cancellous bone 17. In applications where implantation of the structure will be a temporary restorative measure, the surface is prepared to avoid incorporation of and to reduce the adhesion of cancellous bone 17 to the deformable structure 49 thereby facilitating extrication and minimizing trauma to the cancellous bone 17.

Due to the wide range of applications for the deformable structure 49, the bone supporting material 83 may include a number of materials that are selected based on the underlying purpose of the treatment. Where the treatment is for permanent bone support, the bone supporting material 83 includes a cement-like material, such as the previously described PMMA formulation, that may be injected as a liquid, paste or gel, and then permitted to cure or harden within a short period of time. Because the cement-like material is contained and maintained within the deformable structure 49, a wider range of cement-like materials is possible, as the material would not encounter the same biochemical environment as faced by uncontained applications.

In instances where the treatment is for the temporary support of the bone 17, the bone supporting material 83 is injected as a liquid, remains a liquid during the time required for support, and then can be readily withdrawn when the procedure has been completed. In alternative embodiments, the bone supporting material 83 may be in the form of a pliable gel-like material to provide support and energy attenuation for the bone structure.

In applications where disease is a contributing or underlying cause of the bone fracture, a further object of the present invention contemplates that the deformable structure 49 serves as a carrier for treatment of the disease. The aspects of the invention contemplated herein include medicinal, radiological or thermal treatments for the underlying disease condition.

In cases of medicinal treatment regimens, the surface of the metallic film material may be impregnated or coated with a time-release medication targeting the specific disease condition from within the bone itself. Alternatively, the medication may be diffused through a semi-permeable biocompatible material selected for the structure 49 to treat a disease or injury of the bone 17.

In the case of radiological treatment, the radiological treatment is admixed with the bone supporting material 83 by introducing the admixture into the deformable structure 49, such that it is contained and maintained within the deformable structure 49. In this case, the radiological treatment could be withdrawn from the deformable structure 49, after the appropriate exposure to cancellous bone tissue 17 has been attained. Moreover, as the present invention contemplates temporary implantation of the structure 49, it may also be replaced during radiological treatments or after the completion of all radiological procedures.

The thermal treatment may be provided in the first instance as the bone supporting material 83 is introduced into the deformable structure 49. The temperature of the bone supporting material 83 may be adjusted to a desired level prior to introduction into the deformable structure 49. Alternatively, the appropriate temperature may be attained by catalytic reaction of the selected bone supporting material 83. Re-treatment of the bone tissue 17 may be made by subsequent withdrawal and reintroduction of the selected treatment regimen described herein.

FIGS. 8 and 9 depict fixation 100 of a long bone by placing the inventive deformable structure 106 in an intramedullary canal, expanding the deformable structure 106 to draw fracture 102 into a fixed position, then detaching catheter 104 from the deformable structure 106 by decoupling fastener 108 therebetween. In the case of long bone fixation, the deformable structure 106 is preferably an elongate structure capable of anchoring within an intramedullary canal of each bone section being joined. The elongate structure may be placed over a guidewire 101 or may be placed without a guidewire within the intramedullary space.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A device for treating diseased or injured bone, the device having a catheter, wherein the catheter comprises a main body defining at least one interior passage therethrough, an expandable deformable structure, wherein the expandable deformable structure defines an interior space, and a fastener that removably connects the catheter to the expandable deformable structure, wherein the improvement comprises the expandable deformable structure comprised of a metallic material.

2. The device of claim 1, wherein the metallic material is composed of a shape memory metallic material or superelastic metallic material.

3. The device of claim 2, wherein the shape memory metallic material or the superelastic metallic material further comprises a nickel-titanium alloy.

4. A minimally invasive medical device having a deformable structure expandable from a first reduced geometric state to a second expanded geometric state and having a fillable chamber within the deformable structure, a conduit communicating with the fillable chamber, a hardenable filler material capable of being introduced through the conduit and into the fillable chamber, wherein the second expanded geometric shape is supported by the hardenable filler material, wherein the improvement comprises the deformable structure comprising a metal balloon.

5. The device of claim 4, wherein the metal balloon is composed of a shape memory metallic material or superelastic metallic material.

6. The device of claim 5, wherein the shape memory metallic material or the superelastic metallic material further comprises a nickel-titanium alloy.

7. The device of claim 6, wherein the metal balloon further comprises at least one anchoring section thereof.

8. A method of treating diseased or injured bone tissue comprising the steps of:
selecting an interior area in a bone tissue to be treated;
inserting a geometrically expansive metal balloon into the interior area of the bone tissue to be treated;
expanding the metal balloon to a desired expanded three dimensional geometry; and
fixing the desired expanded geometry thereby internally supporting the bone tissue using the device during treatment.

9. The method of claim 8, wherein the selecting step further comprises the step of selecting the interior area using a minimally invasive image-guided technique.

10. The method of claim 8, wherein the interior area in a bone tissue comprises an intramedullary canal.

11. The method of claim 10, wherein the metal balloon further comprises at least one anchoring section thereof.

12. The method of claim 11, wherein the inserting step further comprises the step of inserting the metal balloon into the intramedullary canal over a guidewire.

13. The method of claim 8, wherein the metal balloon is fabricated of a shape memory metallic material or superelastic metallic material.

14. The method of claim 13, wherein the shape memory metallic material or the superelastic metallic material further comprises a nickel-titanium alloy.

15. The method of claim 14, wherein the fixing step further comprises the steps of introducing a filler material into the metal balloon.

16. The method of claim 15, wherein the introducing step further comprises the step of introducing the filler material into the metal balloon through a catheter removably fastened to the metal balloon via a sealable port.

17. The method of claim 16, wherein the introducing step further comprises the steps of:
introducing a fluid having a temperature greater than the phase transition temperature of Nitinol into the metal balloon to raise the temperature of the metal balloon to at least the phase transition temperature of Nitinol and thereby cause the metal balloon to expand; and
introducing the filler material into the expanded metal balloon through the catheter removably fastened to the metal balloon via the sealable port, wherein the filler material comprises a bone supporting material.

18. The method of claim 17 further comprising the steps of:
detaching the catheter from the metal balloon;
sealing the sealable port; and
maintaining the bone supporting material within the metal balloon in a pressurized environment.

19. The method according to claim 18, further comprising the steps of:
reattaching the catheter to the sealable port; and
withdrawing the bone supporting material from the metal balloon.

20. The method according to claim 19, further comprising the steps of:
introducing a fluid having a temperature less than the phase transition temperature of Nitinol into the metal balloon to lower the temperature of the metal balloon to below the phase transition temperature of Nitinol and thereby cause the metal balloon to collapse; and
withdrawing the metal balloon in a collapsed state from the interior area.
